(19) United States
(12) Patent Application Publication
(10) Pub. No.: US 2022/0179987 A1
(43) Pub. Date: Jun. 9, 2022

(54) HEALTH BASED ACCESS CONTROL

(71) Applicant: Carrier Corporation, Palm Beach Gardens, FL (US)

(72) Inventors: Nicholas Venuti, Danville, NH (US); Roger Duchesneau, Stow, MA (US); Kanna Selvakani, Hyderabad (IN); Suzankumar Gongate, Hyderabad (IN)

(21) Appl. No.: 17/544,018

(22) Filed: Dec. 7, 2021

Related U.S. Application Data
(60) Provisional application No. 63/157,949, filed on Mar. 8, 2021.
(60) Provisional application No. 63/199,112, filed on Dec. 7, 2020.

(51) Int. Cl. G06F 21/62; G16H 40/20; G16H 50/30

(52) U.S. Cl. CPC G06F 21/6218; G16H 40/20; G06F 2221/2141; G06F 2221/2145; G16H 50/30

(57) ABSTRACT

A system and method for managing access credentials assigned to a user includes transmitting a self-assessment to a user device associated with a user, and receiving a response to the self-assessment from the user device indicating a condition of the user. The method also can include determining whether to allow or deny the access credentials based at least in part on the response to the self-assessment, and controlling an access device based at least in part on the determination.

### CROSS-REFERENCE TO RELATED APPLICATIONS

This application claims the benefit of U.S. Provisional Application No. 63/199,112 filed Dec. 7, 2020 and U.S. Provisional Application No. 63/157,949 filed Mar. 8, 2021, the disclosures of which are incorporated herein by reference in their entireties.

### BACKGROUND

The present invention relates to access control systems, and more specifically, to the management of access credentials based on the health condition of the person to whom the access credentials are assigned (which may be referred to as a cardholder).

Controlling facility access based on a cardholder's self-assessment is conventionally a manual process whereby the cardholder performs a self-assessment, and an administrator or security officer manually reviews responses and changes cardholder's access (e.g., by disallowing the access credentials from being able to be used to access a protected area, such as an office building, etc.) based on those responses. This manual updating of access rights is inefficient. Accordingly, there remains a need for a way to reduce the administrative burden of managing access credentials based on a cardholder's self-assessment.

### BRIEF DESCRIPTION

According to an embodiment, a method for managing access credentials assigned to a user is provided. The method can include transmitting a self-assessment to a user device associated with a user, and receiving a response to the self-assessment from the user device indicating a condition of the user. The method can also include determining whether to allow or deny the access credentials based at least in part on the response to the self-assessment.

In addition to one or more of the features described herein, or as an alternative, further embodiments include controlling an access device based at least in part on the determination.

In addition to one or more of the features described herein, or as an alternative, further embodiments include using a self-assessment that is a digital form having one or more questions to assess the health condition.

In addition to one or more of the features described herein, or as an alternative, further embodiments include determining whether to allow or deny the access credentials by obtaining the pre-defined criteria for the self-assessment; and comparing the predefined criteria to the response to the self-assessment.

In addition to one or more of the features described herein, or as an alternative, further embodiments include controlling the access device by transmitting a signal to the access device to allow the access credentials if the comparison indicates the predefined criteria of the self-assessment were met.

In addition to one or more of the features described herein, or as an alternative, further embodiments include controlling the access device by transmitting a signal to the access device to deny the access credentials if the comparison indicates the predefined criteria of the self-assessment were not met.

In addition to one or more of the features described herein, or as an alternative, further embodiments include continuously monitoring the health condition with the user device.

In addition to one or more of the features described herein, or as an alternative, further embodiments include updating the access credentials based at least in part on the monitored health condition.

In addition to one or more of the features described herein, or as an alternative, further embodiments include transmitting a notification comprising a status of the health condition to the user device.

In addition to one or more of the features described herein, or as an alternative, further embodiments include a notification that has instructions to route the user device to at least one of an isolation zone or an exit.

According to another embodiment, an access control system is shown. The access control system can include an access controller that has a processer and an access device coupled to the processor. The processor is configured to transmit a self-assessment to a user device associated with a user; receive a response to the self-assessment from the user device indicating a health condition of the user; and determine whether to allow or the deny access credentials assigned to the user based at least in part on the response to the self-assessment.

In addition to one or more of the features described herein, or as an alternative, further embodiments include the processor is further configured to control the access device based at least in part on the determination.

In addition to one or more of the features described herein, or as an alternative, further embodiments include using a self-assessment that is a digital form having one or more questions to assess the health condition.

In addition to one or more of the features described herein, or as an alternative, further embodiments include determining whether to allow or deny the access credentials using a processor that is further configured to obtain pre-defined criteria for the self-assessment; and compare the predefined criteria to the response to the self-assessment.

In addition to one or more of the features described herein, or as an alternative, further embodiments include controlling the access device using a the processor that is further configured to transmit a signal to the access device to allow the access credentials if the predefined criteria of the self-assessment were met.

In addition to one or more of the features described herein, or as an alternative, further embodiments include controlling the access device using a processor that is further configured to transmit a signal to the access device to deny the access credentials if the predefined criteria of the self-assessment were not met.

In addition to one or more of the features described herein, or as an alternative, further embodiments include using a processor that is further configured to continuously monitor the health condition with the user device, the user device comprising one or more sensors used to detect the health condition, and abnormal health condition logic configured to identify the health condition using the one or more sensors.

In addition to one or more of the features described herein, or as an alternative, further embodiments include updating the access credentials based at least in part on the monitored health condition.

In addition to one or more of the features described herein, or as an alternative, further embodiments include using a processor that is further configured to transmit a notification comprising a status of the health condition to the user device.

In addition to one or more of the features described herein, or as an alternative, further embodiments include using a health condition that is based at least in part on an identified cough, wherein the cough is detected by the one or more sensors, wherein the one or more sensors comprises at least one of a microphone or an accelerometer.

The foregoing features and elements may be combined in various combinations without exclusivity, unless expressly indicated otherwise. These features and elements as well as the operation thereof will become more apparent in light of the following description and the accompanying drawings. It should be understood, however, that the following description and drawings are intended to be illustrative and explanatory in nature and non-limiting.

### BRIEF DESCRIPTION OF THE DRAWINGS

The following descriptions should not be considered limiting in any way. With reference to the accompanying drawings, like elements are numbered alike:

FIG. 1 depicts a diagram of an architecture for managing access credentials assigned to a user in accordance with one or more embodiments;

FIG. 2 depicts a flowchart of a method for managing access credentials assigned to a user in accordance with one or more embodiments;

FIG. 3 depicts a schematic illustration of a user device that can be used for completing a self-assessment and monitoring a health condition of the user in accordance with one or more embodiments;

FIG. 4 depicts a flowchart of an exemplary embodiment of monitoring a health condition, a coughing condition, of the user in accordance with one or more embodiments; and

FIGS. 5A and 5B depict flowcharts for operating the access control system in accordance with one or more embodiments.

### DETAILED DESCRIPTION

In one or more embodiments, various technologies can be used to assess or detect symptoms of an individual that may be associated with a viral or bacterial infection. The technologies can be leveraged to receive user inputs that are used to assess an individual's health condition. For example, the inputs may include answers to a questionnaire (which may be referred to herein as a self-assessment) or inputs provided from sensors (e.g., from a user's mobile device, etc.) used to detect symptoms that may be characteristic of different conditions. Additionally, analytics can be performed on the received input to determine if it is likely the individual is infected and should distance themselves from others.

In some embodiments, the self-assessment can be used by an access control system of a premises such as a hospital, office building, retail establishment, etc. to determine whether the individual should be allowed to enter the premises (and/or when a particular individual should be isolated or exit the premises). In different embodiments, the user device (which may be a mobile device, etc.) can be used to monitor and detect the health condition of the user and provide a notification to the user and/or the access control system upon detection of a likely infected condition. The techniques of one or more embodiments described herein provide automated solutions for managing access credentials. For example, by requiring a self-assessment, the health condition of a user can be properly identified and their access credentials (which are associated with the user and stored the memory of the access control system) may be controlled accordingly (i.e., allowed or denied). This may remove the need for manual intervention by an administrator or security officer, who traditionally must manually review responses and changes cardholder's access.

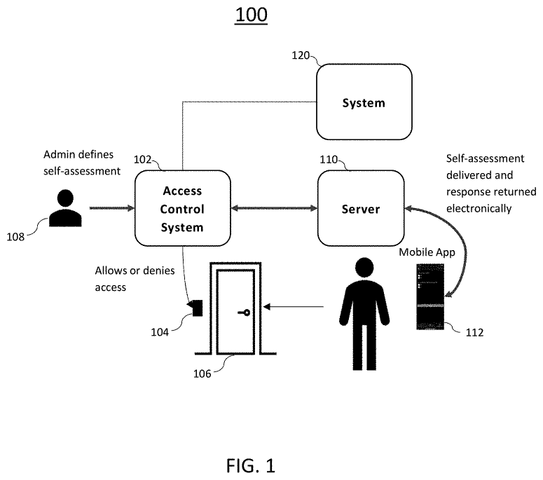

FIG. 1 depicts a block diagram for an architecture 100 for managing access credentials assigned to a user. As shown, the architecture 100 includes an access control system 102 in accordance with one or more embodiments. The access control system 102 can include a controller, processor, or computing unit.

The access control system 102 can be coupled to one or more access devices 104, such as an electronic locking mechanism. The access devices 104 are used to restrict access points 106 such as a door, gate, elevator, etc. that are used to limit admission to one or more areas of a building. Although a single access device 104 and access point 106 are shown in FIG. 1, it should be understood that a plurality of access devices 104 and corresponding access points 106 can be used and is not intended to be limited by the illustration. To access a particular access point 106 a user may have to present access credentials to the access device 104 assigned to the particular access point 106. For example, access credentials may be presented using a card that is swiped or placed within proximity to the access device 104. In another example, the access credentials can be presented by interacting with a keypad or biometric scanner, etc. In a further embodiment, the access credentials may be transmitted from a user device to a server 120 or access control system 102 to determine the appropriate level of access. If the access control system 102 determines that the access credentials are allowed then the access points 106 can be unlocked access to allow a user to gain access. It should be appreciated that the decision making for determining of whether or not the presented access credentials are allowed or denied may be made in any suitable processor, which may be localized or remote (e.g., cloud-based).

The access control system 102 can be coupled to a server 110. Although the server 110 is shown external to the access control system 102, it should be understood the functionality of the server 110 can be integrated into the access control system 102 or a different architecture can be used to distribute the functionality of the system 100. The access control system 102 can be coupled to the server 110 over a network. The networks can include any type of network, for example, the Internet, a local area network, a wide area network and/or a wireless network. The network can comprise copper transmission cables, optical transmission fibers, wireless transmission, routers, firewalls, switches, gateway computers, and/or edge servers. The server 110 can be configured to communicate with one or more user devices 112, such as a tablet, mobile phone, or other computing device, associated with a user that desires to gain access to the restricted area. The server 110 can also be configured to communicate with other devices such as dedicated kiosks that allow a user to provide their access credentials to gain access to the restricted areas 106.

The server 110 can be configured to transmit web-based or mobile-based prompts to the user device 112 responsive to detecting the presence of the user. The presence of the user can be detected in a variety of ways including using the location information on the user device 112 or using presence detection sensor or camera (not shown). The self-assessment can be a digital form and can displayed on the user device 112 (e.g., in a mobile application downloaded and stored on the user device 112).

In one or more embodiments, an administrator 108 can define the self-assessment to include one or more questions, prompts, or challenges, using a computing system (not shown). The self-assessment can include any number of questions such as 3, 5, 10, etc. These pre-defined criteria can be used to determine whether the user should be granted access. For example, such predefined criteria may be assessed by questions that are related to how the user is feeling including whether the user has a fever, feeling unusually tired or achy, nauseous, sore throat, loss of taste/smell, or any other conditions that may be indicative of an illness, etc. In addition, the questions can be related to the user's contact with others suspected of having an infection.

In one or more embodiments, the user can provide a response to the questions using a web-based or mobile application on the user device 112 which may transmit the response to the access control system 102. In some embodiments, the input from the user device 112 may be transmitted to the server 110. The server 110 can provide the data in the appropriate format between the access control system 102 and the user device 112.

The access control system 102 can compare the received responses to the administrator-defined self-assessment to determine the level of user access that should be granted to the user associated with the user device 112. The access control system 102 can be configured to require that all of the questions be answered correctly or that a majority of the questions be answered correctly to indicate that a user should be allowed access to the building (i.e., whether or where the user's access credentials may be allowed or denied). The pre-defined criteria can map an acceptable answer to each of the questions. The responses to the questions can include “yes” and “no” responses. The responses may also include a numerical response such as a number that falls within a range such as between 98.0-98.8 representing the normal body temperature.

In some embodiments, the access control system 102 can restrict or deny access credentials assigned to a user whose responses failed to meet the predefined criteria and electronically allow access credentials assigned to a user whose responses met the predefined criteria. If the user access is granted, the access control system 102 can transmit a signal to the access device 104 to unlock the access point 106. Alternatively, the access control system 102 can restrict the user access by keeping the access point in a locked position.

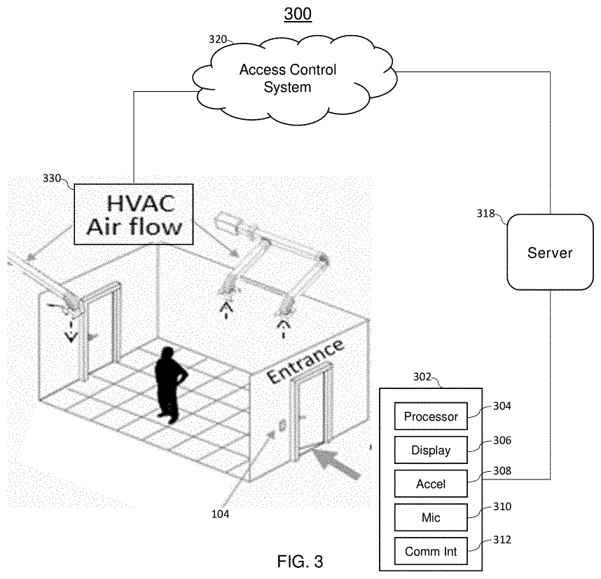

FIG. 1 can also include additional systems 120 that may be coupled to and controlled by the access control system 102 or server 110. In a non-limiting example, the system 120 can include an HVAC system such as that discussed with reference to FIG. 3. It should be appreciated that the system 120 can include a plurality of interconnected systems (e.g., a security system, a building services systems, etc.).

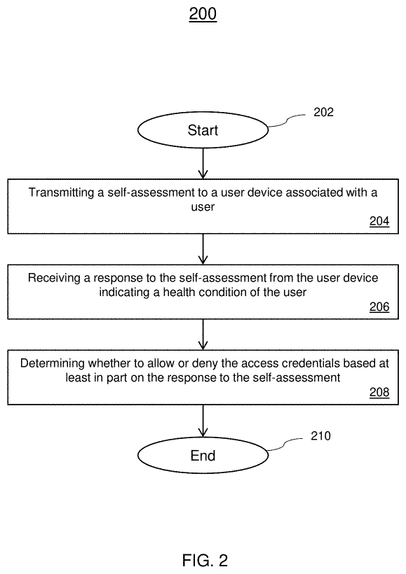

FIG. 2 depicts a flowchart of method 200 for managing access credentials assigned to a user in accordance with one or more embodiments. The method 200 can be performed using the system 100 shown in FIG. 1 in certain instances. It is envisioned that other suitable system configurations may be used in other instances. In one or more embodiments, the processor of the access control system 102 performs the following process steps. The method 200 begins block 202 and proceeds to block 204 which provides for transmitting a self-assessment to a user device 112 associated with a user. It will be appreciated that the association of a user device 112 to a user may be completed by storing identifiable information about the respective user (e.g., name, employee number, etc.) and the user device 112 (e.g., product serial number, phone number, etc.) in the access control system 102 and/or the server 120. Block 206 receives a response to the self-assessment from the user device 112 indicating a health condition of the user. Block 208 determines whether to allow or deny the access credentials based at least in part on the response to the self-assessment. The method 200 ends at block 210. It should be understood that a different sequence of steps or additional steps can be incorporated into the method 200.

One or more illustrative embodiments of the disclosure are described herein. Such embodiments are merely illustrative of the scope of this disclosure and are not intended to be limiting in any way. Accordingly, variations, modifications, and equivalents of embodiments disclosed herein are also within the scope of this disclosure.

FIG. 3 depicts a schematic illustration of a system 300 incorporating a user device that can be used for completing a self-assessment and monitoring a health condition of the user. The user device 302 can be used to continuously detect an abnormal health condition in certain instances. It will be appreciated that the health condition of the user can be monitored prior to reaching a destination or after the user has been granted access to the building. For example, various symptoms may be monitored, such as a cough, heart rate, etc. which can be indicative of a health condition of the user.

The results of the detection can be transmitted to an access control system 320, using a communication interface 312. In certain instances the user device is used to continuously monitor the health condition (which may be transmitted to the access control system 320 from time to time to update the user's status and level of access). In some embodiments, a server 318 functions as an intermediary between the user device 302 and access control system 320.

In one or more embodiments, the user device 302 can be configured with a mobile application that uses the sensor data obtained from one or more sensors equipped on the user device. The mobile application can perform data analytics on the sensor data to determine whether the health condition is characteristic of an infected person.

In a non-limiting example, the health condition can include a user's cough where an accelerometer can be used to capture the coughing vibrations and the microphone can be used to capture the coughing sounds (measured in decimal values). An infected cough can be indicative of an unhealthy user.

In one or more embodiments, a processor 304 of the user device 302 can be configured (e.g., using an application stored on the user device 302) with abnormal health detection logic such as a cough detection logic to detect a coughing rate or coughing frequency of the user measured by one or more sensors equipped on the user device. These sensors can include, but are not limited to, a microphone (mic) 310 and/or an accelerometer (accel) 308. It should be understood the sensors are not limited by the examples and the user device 302 can include additional sensors or use different types of sensors based on the condition that is being monitored. For example, biometric sensors (which may be pair to the user device 302, such as a smartwatch, etc.) can be used to obtain heart rate information, etc.

The cough detection logic can be used to distinguish between a normal cough and an infected cough. In some scenarios, the frequency of coughs that occur within a period of time can provide an indication of an infected cough or abnormal condition. The detected cough can be compared to a predefined pattern of cough profiles to determine whether the detected cough is indicative of an infection. The pattern can include vibration information, frequency information, and/or the amplitude information of the associated cough.

In one or more embodiments, the cough detection logic can store information related to predefined coughing sounds that is used to identify infected coughs. In addition, the cough detection logic can also store information for various coughing patterns. The predefined coughing sounds and predefined coughing patterns can be used to identify infected coughs of an unhealthy person. For example, the coughing sounds can include deep coughs, shallow coughs, congestion-filled coughs, etc. Also, coughing patterns can include coughs that occur in rapid succession, coughs that are clustered in groups, etc. This can be indicative of the user's health.

In another embodiment, the detected coughing pattern can be compared to the user's normal pattern of coughing or an average coughing pattern for the user. In this scenario, the user's historical pattern of coughing can be tracked over a period of time.

It should be understood that other types of abnormal health conditions can be monitored by the user device 302. In another embodiment, an abnormal health condition may consider a user's temperature or sinus/chest congestion or another type of measurable symptom that can be detected by biometric sensors or audio devices of the user device 302.

The user device 302 can also include a display 306 that is used to provide notifications to the user. In addition, the display 306 can be used as an input interface, such as a touch screen interface, to receive user inputs (e.g., for completing the self-assessment, etc.).

In one or more embodiments, if an infected cough is identified by the user device 302 after a user has entered a building, the access control system 320 can modify the user access by restricting the user access within the building. For example, one or more doors that are in proximity to the user and are controlled by an access device can be locked.

Also, the access control system 320 can transmit a door locked message to the user device 302. In a different embodiment, the notification can provide instructions for the user to exit the building or head towards an isolation zone. An isolation zone is an area that is reserved for individuals that are exhibiting symptoms that are associated with an unhealthy condition. The isolation zone can be restricted from other users who are not authorized to be in the isolation zone. In some embodiments, the user device 302 can be provided specific instructions to route the user to the isolation zone or to an exit. This can reduce the user's exposure and potential spread to others in the building.

In some embodiments, the operation of the HVAC system 330 can be modified to reduce the potential spread of a virus. For example, responsive to the identification of an infected person, the HVAC system 330 can receive a control signal from the access control system 320 to reduce the air flow in one or more zones of a building. In another example, the HVAC system 330 can redirect and/or vent the air flow to the outdoors so the internal air is not recycled within the building.

In a further example, the HVAC system 330 can increase or decrease the air flow based on the detected abnormal condition and the configuration of the building. The operation of the HVAC system 330 is not intended to be limited by the examples described herein but are merely an example illustration.

The access control system 320 can also be configured to communicate with other systems/devices. For example, upon identification of a potentially infected individual the access control system 320 can be configured to notify the proper medical and security personnel of the situation. The appropriate staff may then assist the individual to exit the building or provide the appropriate level of care. In a non-limiting example, the staff can receive the location of the isolation zone for the user. In the event that medical staff is not located on site, the access control system can provide details for the closest medical service or hospital service.

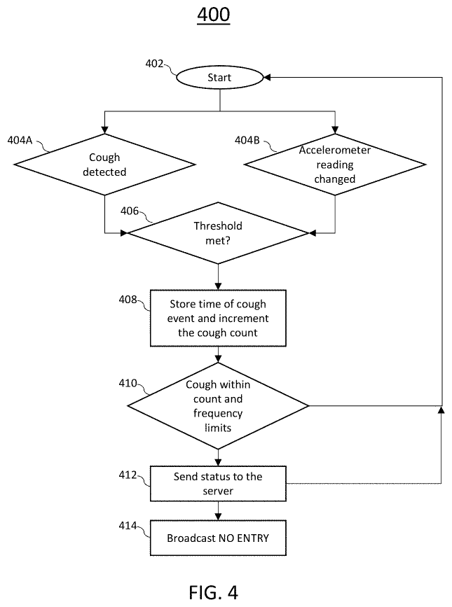

FIG. 4 depicts a flowchart of a method 400 for detecting a health condition in accordance with one or more embodiments. In a non-limiting example, the health condition can include an infection caused by a virus or bacteria, where symptoms can include a cough that is indicative of an infection or virus. The method 400 begins at block 402 and proceeds to decision blocks 404A and 404B. Block 404A determines whether the user device has detected a cough and decision block 404B determines whether an accelerometer reading of the user device has changed. The processing steps at block 404A and 404B can be performed simultaneously or sequentially, and is not intended to be limited by FIG. 4. At block 406, it is determined whether the detected cough and the accelerometer reading exceed a configurable threshold. The configurable threshold related to the cough can be a cough frequency rate and the threshold associated with the accelerometer reading can be associated with a defined pattern of vibrations or a change in the detected vibration.

At block 408, the method 400 can store the time that a cough event is detected by the user device and increments the cough count appropriately. At block 410, the method 400 determines whether the detected coughs are within the cough count and cough frequency limits. If so (“Yes” branch), the method 400 returns to the beginning of the process and continues to monitor the user. If the detected coughs exceed the cough count threshold and/or the cough frequency, the method 400 continues to block 412 and sends a status to the server. At block 414, a NO ENTRY message is broadcasted to the user device.

One or more illustrative embodiments of the disclosure are described herein. Such embodiments are merely illustrative of the scope of this disclosure and are not intended to be limiting in any way. Accordingly, variations, modifications, and equivalents of embodiments disclosed herein are also within the scope of this disclosure.

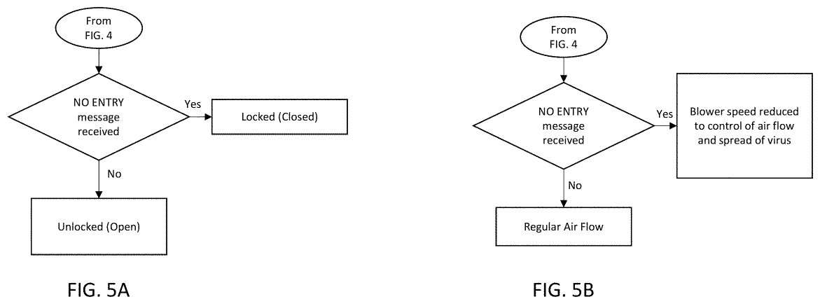

FIG. 5A depicts a process flow that if a NO ENTRY message is transmitted to a user device 302, the access device 104 is locked or closed to restrict access to the user. Otherwise, if the NO ENTRY message is not received, the access device 104 can be unlocked or opened.

FIG. 5B depicts a process flow that if a NO ENTRY message is received the blower speed of the HVAC system 320 can be reduced to control the air flow and reduce the spread of the virus. Otherwise, if the NO ENTRY message is not received, the air flow operates normally without modification. It should be understood the process flow for FIGS. 5A and 5B can occur simultaneously or sequentially.

The technical effects and benefits include eliminating the need for intermediary personnel to manually review each of the answers provided from the self-assessment of an individual that would like to enter the premises. The review of the self-assessment and determination for the level of access are automated and can increase the processing efficiency of the self-assessment.

Also, the technical effects and benefits include the early identification of symptoms associated with a virus using analytics. This self-identification of potentially contagious conditions prior to the individuals coming into contact with other users can provide great advantages. This can reduce the chance of infection to those that are within proximity of the individual. The techniques described herein enable early mitigation for potential unhealthy persons.

A detailed description of one or more embodiments of the disclosed apparatus and method are presented herein by way of exemplification and not limitation with reference to the Figures.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof

What is claimed is:

1. A method for managing access credentials assigned to a user, the method comprising:
transmitting, using a processor, a self-assessment to a user device associated with a user;
receiving, at the processor, a response to the self-assessment from the user device indicating a health condition of the user; and
determining, using the processor, whether to allow or deny the access credentials based at least in part on the response to the self-assessment.

2. The method of claim 1, further comprising controlling, using the processor, an access device based at least in part on the determination.

3. The method of claim 1, wherein the self-assessment is a digital form comprising one or more questions to assess the health condition.

4. The method of claim 2, wherein determining whether to allow or deny the access credentials comprises:
obtaining pre-defined criteria for the self-assessment; and
comparing the predefined criteria to the response to the self-assessment.

5. The method of claim 4, wherein controlling the access device comprises transmitting a signal to the access device to allow the access credentials if the comparison indicates the predefined criteria of the self-assessment were met.

6. The method of claim 4, wherein controlling the access device comprises transmitting a signal to the access device to deny the access credentials if the comparison indicates the predefined criteria of the self-assessment were not met.

7. The method of claim 1, further comprising continuously monitoring the health condition with the user device.

8. The method of claim 7, further comprising updating the access credentials based at least in part on the monitored health condition.

9. The method of claim 7, further comprising transmitting a notification comprising a status of the health condition to the user device.

10. The method of claim 9, wherein the notification comprises instructions to route the user device to at least one of an isolation zone or an exit.

11. An access control system comprising:
an access controller comprising a processer;
an access device coupled to the processor, wherein the processer is configured to:
transmit a self-assessment to a user device associated with a user;
receive a response to the self-assessment from the user device indicating a health condition of the user; and
determine whether to allow or deny or deny access credentials assigned to the user based at least in part on the response to the self-assessment.

12. The access control system of claim 11, wherein the processor is further configured to control the access device based at least in part on the determination.

13. The access control system of claim 11, wherein the self-assessment is a digital form comprising one or more questions to assess the health condition.

14. The access control system of claim 12, wherein determining whether to allow or deny the access credentials comprises the processor being further configured to:
obtain pre-defined criteria for the self-assessment; and
compare the predefined criteria to the response to the self-assessment.

15. The access control system of claim 14, wherein controlling the access device comprises the processor being further configured to transmit a signal to the access device to allow the access credentials if the predefined criteria of the self-assessment were met.

16. The access control system of claim 14, wherein controlling the access device comprises the processor being further configured to transmit a signal to the access device to deny the access credentials if the predefined criteria of the self-assessment were not met.

17. The access control system of claim 1, wherein the processor is further configured to continuously monitor the health condition with the user device, the user device comprising one or more sensors used to detect the health condition, and abnormal health condition logic configured to identify the health condition using the one or more sensors.

18. The access control system of claim 17, further comprising the processor being configured to update the access credentials based at least in part on the monitored health condition.

19. The access control system of claim 17, wherein the processor is further configured to transmit a notification comprising a status of the health condition to the user device.

20. The access control system of claim 17, wherein the health condition is based at least in part on an identified cough, wherein the cough is detected by the one or more sensors, wherein the one or more sensors comprises at least one of a microphone or an accelerometer.
